(19) United States
(12) Patent Application Publication
(10) Pub. No.: US 2008/0293616 A1
(43) Pub. Date: Nov. 27, 2008

(54) Use of 2-(1,1,4-Trimethylpent-3-Enyl)-4,7-Dihydro-1,3-Dioxepine

(76) Inventors: Erich Dilk, Holzminden (DE); Horst Surburg, Holzminden (DE); Heinz-Jurgen Bertram, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 11/597,510

(22) PCT Filed: Feb. 14, 2005
(86) PCT No.: PCT/EP05/50630

§ 371 Date: Sep. 18, 2007

(51) Int. Cl. A61K 8/49; A61Q 13/00

(52) U.S. Cl. 512/12

(57) ABSTRACT

The use of 2-(1,1,4-trimethylpent-3-enyl)-4,7-dihydro-1,3-dioxepin as an agent for providing (a) hair or (b) textile fibres with a fresh smell is described.

The invention relates to novel uses of the cyclic acetal 2-(1,1,4-trimethylpent-3-enyl)-4,7-dihydro-1,3-dioxepin and mixtures containing this compound.

Compounds of the acetal class have been described in large numbers and often have a pleasant odour, so they are used inter alia as fragrances. A survey of fragrances with an acetal structure has been made e.g. by G. Buchbauer and C. Lux in Parfümerie und Kosmetik, 72, 792 (1991).

The use as fragrances of acetals with a 4,7-dihydro-1,3-dioxepin structure, resulting from the reaction of aldehydes or ketones with cis-2-butene-1,4-diol, has been described relatively rarely. U.S. Pat. No. 3,822,291 mentions 2-(3-pentyl)-4,7-dihydro-1,3-dioxepin (carotene), which is said to have a natural, rooty, earthy odour. U.S. Pat. No. 3,936,398 describes 2-(3-methylbutyl)-2-methyl-4,7-dihydro-1,3-dioxepin (amber sage) as having a fresh flowery scent of linalool and coriander character. This dioxepin is mentioned not only for use in fine perfumery but also for use in the perfuming of soaps and detergents.

Among other compounds, DE 195 32 318 lists 2-(1,1,4-trimethylpent-3-enyl)-4,7-dihydro-1,3-dioxepin, which is said to have a fresh, fruity and at the same time woody note. Apart from use in fine perfumery, however, this compound is only mentioned as being useful e.g. for the perfuming of soaps, cleaning agents and detergents or fabric softeners.

Fragrance mixtures with a particularly fresh-green top note, which simultaneously have a pronounced blooming (odour from an aqueous surfactant solution), are often sought after, especially for the perfuming of formulations containing surfactants, such as shampoos, detergents or fabric softeners. Another important application technology requirement of fragrance mixtures for products containing surfactants is their substantivity towards or retention on the substrate, especially hair or textile fibres. The meaning of substantivity and retention is explained in detail e.g. in EP 1 201 738 A1, cf. sections [0004]-[0005]. In general, therefore, fragrances with a high substantivity and/or retention are also sought after.

A first feature of the present invention, which is closely connected with the general objects mentioned above, relates to the use of 2-(1,1,4-trimethylpent-3-enyl)-4,7-dihydro-1,3-dioxepin as an agent for providing (a) hair or (b) textile fibres with a fresh smell.

Previous patent application DE 195 32 318 A1 makes no reference to the particularly high substantivity and retention of 2-(1,1,4-trimethylpent-3-enyl)-4,7-dihydro-1,3-dioxepin, which is surprising in comparison with structurally related substances and is responsible for the fact that the use according to the invention produces outstanding results. In fact, hair and textile fibres which have been treated with 2-(1,1,4-trimethylpent-3-enyl)-4,7-dihydro-1,3-dioxepin have a fresh smell that persists for a surprisingly long time and is also particularly resistant to washing (with water).

In accordance with these findings, a further feature of the invention relates to the use of 2-(1,1,4-trimethylpent-3-enyl)-4,7-dihydro-1,3-dioxepin as an agent for increasing the substantivity and/or retention of a fragrance mixture (towards or on hair or textile fibres). By adding 2-(1,1,4-trimethylpent-3-enyl)-4,7-dihydro-1,3-dioxepin to a given fragrance mixture of only low substantivity and/or retention, these properties are improved in a particularly advantageous manner. Thus, for example, by the addition of 2-(1,1,4-trimethylpent-3-enyl)-4,7-dihydro-1,3-dioxepin, an aqueous washing solution (or a corresponding detergent or shampoo or the like) that smells fresh but, because of the inadequate substantivity of the perfumes it contains, is not suitable for passing on a fresh odour to fabrics (textile fibres) or hair can be converted to a solution that is outstanding at passing on a fresh odour which persists for a long time on the treated substrates (hair or textile fibres).

A corresponding process according to the invention for providing (a) hair or (b) textile fibres with a fresh smell comprises the following steps:
preparation of a mixture containing 2-(1,1,4-trimethylpent-3-enyl)-4,7-dihydro-1,3-dioxepin, and
application of the mixture to the hair or textile fibres.

A solution according to the invention which is particularly suitable for the uses according to the invention or the corresponding processes comprises (a) water, (b) 2-(1,1,4-trimethylpent-3-enyl)-4,7-dihydro-1,3-dioxepin and (c) one or more surfactants, the concentration of 2-(1,1,4-trimethylpent-3-enyl)-4,7-dihydro-1,3-dioxepin in the solution being in the range from 10⁻⁷ to 10⁻¹ wt. %. Other fragrances and/or miscellaneous conventional additives can be present.

DE 19532318 A1, cited above, makes no reference to the particular olfactory effects of 2-(1,1,4-trimethylpent-3-enyl)-4,7-dihydro-1,3-dioxepin in the context of aqueous products containing surfactants.

Surprisingly, in the uses, processes and solutions according to the invention, the use of 2-(1,1,4-trimethylpent-3-enyl)-4,7-dihydro-1,3-dioxepin achieves a fresh-green top note (including interesting chrysanthemum and grapefruit nuances) not only in conjunction with a high substantivity/retention but also in conjunction with a surprising blooming (i.e. the odour perceived above an aqueous solution containing surfactants). This combination of sought-after properties could not be found in the state of the art or, in particular, in DE 195 32 318 A1. As the Examples below show in detail, particularly the compounds from DE 195 32 318 which are structurally very similar to 2-(1,1,4-trimethylpent-3-enyl)-4,7-dihydro-1,3-dioxepin (Examples 3-5 of said patent) do not exhibit this fortunate combination of properties any more than amber sage or 2-(1,1,4-trimethyl-3-pentenyl)-1,3-dioxane.

Accordingly, a further feature of the invention relates to the use of 2-(1,1,4-trimethylpent-3-enyl)-4,7-dihydro-1,3-dioxepin for increasing the odour perceived above an aqueous solution containing surfactants (e.g. a wash liquor), i.e. for increasing the blooming.

The desired fresh-green top note with a pronounced blooming and an increased substantivity for aqueous applications involving surfactants can normally be achieved by using only a small dose of 2-(1,1,4-trimethylpent-3-enyl)-4,7-dihydro-1,3-dioxepin in a resulting perfume composition.

2-(1,1,4-Trimethylpent-3-enyl)-4,7-dihydro-1,3-dioxepin can be used in a large number of products. The pH of an aqueous formulation containing the acetal to be used according to the invention is preferably not lower than 6, particularly for stability reasons; a pH of >8 is preferred.

Examples of fragrances with which 2-(1,1,4-trimethylpent-3-enyl)-4,7-dihydro-1,3-dioxepin can advantageously be combined can be found e.g. in S. Arctander, Perfume and Flavor Materials, Vol. I and II, Montclair, N.J., 1969, Selbstverlag, or K. Bauer, D. Garbe and H. Surburg, Common Fragrance and Flavor Materials, 4th Ed., Wiley-VCH, Weinheim 2001.

The following may be specifically mentioned:

extracts of natural raw materials, such as ethereal oils, concrete oils, absolute essences, resins, resinoids, balsams, tinctures, e.g.:

ambergris tincture; amyris oil; angelica seed oil; angelica root oil; anise oil; baldrian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill oil; dill seed oil; eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacum wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; Scotch fir oil; spearmint oil; caraway oil; ladanum oil; ladanum absolute; ladanum resin; lavandin absolute; lavandin oil; lavender absolute; lavender oil; lemongrass oil; lovage oil; distilled lime oil; pressed lime oil; linaloa oil; litsea cubeba oil; laurel leaf oil; mace oil; marjoram oil; mandarin oil; massoi bark oil; mimosa absolute; ambrette oil; musk tincture; muscatel sage oil; nutmeg oil; myrrh absolute; myrrh oil; myrtle oil; clove leaf oil; clove blossom oil; neroli oil; frankincense absolute; frankincense oil; opopanax oil; orange blossom absolute; orange oil; oreganum oil; palmarosa oil; patchouli oil; perilla oil; Peruvian balsam oil; parsley leaf oil; parsley seed oil; petitgrain oil; peppermint oil; pepper oil; pimento oil; pine oil; European pennyroyal oil; rose absolute; rosewood oil; rose oil; rosemary oil; Dalmatian sage oil; Spanish sage oil; sandalwood oil; celery seed oil; lavender spike oil; Japanese anise oil; styrax oil; tagetes oil; fir needle oil; tea tree oil; turpentine oil; thymian oil; tolu balsam; tonka absolute; tuberose absolute; vanilla extract; violet leaf absolute; verbena oil; vetiver oil; juniper oil; wine yeast oil; wormwood oil; wintergreen oil; ylang oil; hyssop oil; civet absolute; cinnamon leaf oil; cinnamon bark oil; and fractions thereof or ingredients isolated therefrom;

individual fragrances from the following groups:

hydrocarbons, e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

aliphatic alcohols, e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-¾-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

aliphatic aldehydes and their acetals, e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyloxyacetaldehyde;

aliphatic ketones and their oximes, e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

aliphatic sulfur-containing compounds, e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

aliphatic nitrites, e.g. 2-nonenoic acid nitrile; 2-tridecenoic acid nitrile; 2,12-tridecadienoic acid nitrile; 3,7-dimethyl-2,6-octadienoic acid nitrile; 3,7-dimethyl-6-octenoic acid nitrile;

aliphatic carboxylic acids and their esters, e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynate; methyl 2-nonynate; allyl 2-isoamyloxyacetate; methyl 3,7-dimethyl-2,6-octadienoate;

acyclic terpene alcohols, e.g. citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylen-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and their formates, acetates, propionates, isobutyrates, butyrates, isovalerates, pentanoates, hexanoates, crotonates, tiglates, 3-methyl-2-butenoates;

acyclic terpene aldehydes and ketones, e.g. geraniol; nerol; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;
cyclic terpene alcohols, e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and their formates, acetates, propionates, isobutyrates, butyrates, isovalerates, pentanoates, hexanoates, crotonates, tiglates, 3-methyl-2-butenoates;
cyclic terpene aldehydes and ketones, e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);

cyclic alcohols, e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

cycloaliphatic alcohols, e.g. alpha-3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

cyclic and cycloaliphatic ethers, e.g. cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;

cyclic and macrocyclic ketones, e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;

cycloaliphatic aldehydes, e.g. 2,4-dimethyl-3-cyclohexenecarbaldehyde; 2-methyl-4-(2,2,6-trimethylcyclohexen-1-yl)-2-butenal; 4-(4-hydroxy-4-methyl-pentyl)-3-cyclohexenecarbaldehyde; 4-(4-methyl-3-penten-1-yl)-3-cyclohexenecarbaldehyde;

cycloaliphatic ketones, e.g. 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl 2,4-dimethyl-3-cyclohexen-1-yl ketone;

esters of cyclic alcohols, e.g. 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;

esters of cycloaliphatic carboxylic acids, e.g. allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexene-carboxylate; ethyl 2-methyl-1,3-dioxolan-2-acetate;

araliphatic alcohols, e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenyl-propanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

esters of araliphatic alcohols and aliphatic carboxylic acids, e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethyl-phenylethyl acetate; alpha,alpha-dimethylphenylethyl butyrate; cinnamyl acetate; 2-phenoxyethyl isobutyrate; 4-methoxybenzyl acetate;

araliphatic ethers, e.g. 2-phenylethyl methyl ether; 2-phenylethyl isoamyl ether; 2-phenylethyl 1-ethoxyethyl ether; phenylacetaldehyde dimethyl acetal; phenyl-acetaldehyde diethyl acetal; hydratropaldehyde dimethyl acetal; phenylacetaldehyde glyceryl acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

aromatic and araliphatic aldehydes, e.g. benzaldehyde; phenylacetaldehyde; 3-phenylpropanal; hydratropaldehyde; 4-methylbenzaldehyde; 4-methylphenyl-acetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
aromatic and araliphatic ketones, e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

aromatic and araliphatic carboxylic acids and their esters, e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

aromatic nitrogen-containing compounds, e.g. 2,4,6-trinitro-1,3-dimethyl-5-tert-butylbenzene; 3,5-dinitro-2,6-dimethyl-4-tert-butylacetophenone; cinnamic acid nitrile; 5-phenyl-3-methyl-2-pentenoic acid nitrile; 5-phenyl-3-methylpentanoic acid nitrile; methyl anthranilate; methyl N-methylanthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropyl-quinoline; 6-isobutylquinoline; 6-sec-butylquinoline; indole; skatole; 2-methoxy-3-isopropylpyrazine; 2-isobutyl-3-methoxypyrazine;

phenols, phenyl ethers and phenyl esters, e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;

heterocyclic compounds, e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

lactones, e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

Perfume compositions are preferably prepared using combinations with other macrocyclic musk fragrances, e.g. 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene 1,12-dodecanedioate and ethylene 1,13-tridecanedioate.

The amount of 2-(1,1,4-trimethylpent-3-enyl)-4,7-dihydro-1,3-dioxepin used in perfume compositions is 0.05 to 50 wt. %, preferably 0.5 to 20 wt. %, based on the total perfume oil composition.

Perfume oils containing 2-(1,1,4-trimethylpent-3-enyl)-4,7-dihydro-1,3-dioxepin can be used in concentrated form, in solutions or in the modified forms described below for the perfuming of e.g. (acidic), alkaline and neutral cleaning agents such as carpet cleaning powders and foams, liquid detergents, powder detergents, fabric preconditioners like bleach, soaker and stain remover, fabric softeners, washing soaps, washing tablets, body care products such as solid and liquid soaps, shower gels, shampoos, cosmetic emulsions of the oil-in-water, water-in-oil and water-in-oil-in-water type, and hair care products such as hair sprays, hair gels, strengthening hair lotions, hair rinses, permanent and semipermanent hair dyes, hair styling products like cold waving and straightening products, hair tonics, hair creams and hair lotions.

Perfume oils containing the acetal to be used according to the invention can be used in perfumed products in liquid form, either undiluted or diluted with a solvent. Examples of suitable solvents for this purpose are ethanol, isopropanol, diethylene glycol monoethyl ether, glycerol, propylene glycol, 1,2-butylene glycol, dipropylene glycol, diethyl phthalate, triethyl citrate, isopropyl myristate, etc.

Perfume oils containing the acetal to be used according to the invention can also be adsorbed on a carrier to ensure both a fine distribution of the fragrances in the product and a controlled release when applied. Such carriers can be porous inorganic materials such as light sulfate, silica gels, zeolites, gypsums, clays, clay granules, gas concrete, etc., or organic materials such as woods and cellulose-based substances.

Perfume oils containing the acetal to be used according to the invention can also be microencapsulated or spray-dried or in the form of inclusion complexes or extrusion products, and can be added in this form to the product to be perfumed.

Optionally, the properties of the perfume oils modified in this way can be further optimized, in respect of a more specific perfume release, by coating with suitable materials; waxy plastics, e.g. polyvinyl alcohol, are preferably used for this purpose.

Microencapsulation of the perfume oils can be effected for example by the so-called coacervation process with the aid of capsule materials made e.g. of polyurethane-like substances or soft gelatin. Spray-dried perfume oils can be prepared for example by spray drying an emulsion or dispersion containing the perfume oil, it being possible for modified starches, proteins, dextrins and vegetable gums to be used as carriers. Inclusion complexes can be prepared for example by introducing dispersions of the perfume oil and cyclodextrins or urea derivatives into a suitable solvent, e.g. water. Extrusion products can be prepared by melting the perfume oils with a suitable waxy substance and by extrusion with subsequent solidification, optionally in a suitable solvent, e.g. isopropanol.

Preferred products which can be used within the framework of the present invention are (a) perfume oil mixtures for formulations containing surfactants, e.g. cleaning agents, detergents, fabric softeners and body care products, and (b) the corresponding formulations themselves which contain surfactants.

The formulations containing surfactants which can be used within the framework of the present invention generally include substances from the class of anionic surfactants, e.g. carboxylates, sulfates, sulfonates and phosphates, cationic surfactants, e.g. quaternary ammonium salts, amphoteric surfactants, e.g. betaines, and non-ionic surfactants, e.g. ethoxylates and propoxylates.

Preferred anionic surfactants are sulfates and sulfonates. Preferred sulfates are those having 12 to 18 carbon atoms and a degree of ethoxylation of 1 to at most 5. Sodium laurylethersulfate, preferably having a mean degree of ethoxylation of 2 to 4, is particularly preferred.

Particularly preferred sulfonates are linear sodium alkylbenzenesulfonates having an average of approx. 12 carbon atoms in the alkyl chain, said alkyl chains consisting of homologous radicals having 10 to 14 carbon atoms (“dodecylbenzenesulfonate”).

Preferred compounds from the group of non-ionic surfactants are ethoxylated fatty alcohols obtained by the ethoxylation of alcohols having 12 to 18 carbon atoms (fatty alcohol ethoxylates having 12 to 18 C atoms). The degree of ethoxylation here can vary within wide limits, but particularly preferred products are those having an average degree of ethoxylation of 5 to 10 or, in particular, 7 mol of added ethylene oxide per mol of fatty alcohol.

Particularly preferred betaines are those of the acid amide type having the structure shown:

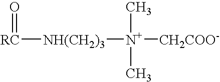

A preferred radical RC═O is the coconut oil fatty acid cut in which lauric acid is the main constituent at 45-50%.

In combination with selected surfactants, the favourable properties of 2-(1,1,4-trimethylpent-3-enyl)-4,7-dihydro-1,3-dioxepin are surprisingly pronounced. A corresponding surfactant formulation according to the invention (mixture according to the invention) comprises 2-(1,1,4-trimethylpent-3-enyl)-4,7-dihydro-1,3-dioxepin and one or more surfactants selected from the group comprising:
linear alkylbenzenesulfonates (especially those mentioned above, e.g. linear sodium alkylbenzenesulfonates),
fatty alcohol ethoxylates having 12-18 C atoms (especially those mentioned above, e.g. those having the degree of ethoxylation identified above as preferred),
laurylethersulfates (especially those mentioned above, e.g. the sodium laurylethersulfate mentioned above) and
betaines (especially those mentioned above, e.g. betaines of the acid amide type having the structure shown above).

Linear alkylbenzenesulfonates and fatty alcohol ethoxylates having 12-18 C atoms are preferably used together with one another here, especially in heavy-duty detergent powders.

Likewise, laurylethersulfates (especially the sodium laurylethersulfate mentioned above) and betaines (especially those of the acid amide type having the structure shown above) are preferably used together with one another, especially in light-duty detergents, shampoos and shower gels.

The concentration of surface-active substances in the surfactant formulations according to the invention is not normally critical. Preferred concentrations depend on the type of surfactant and the particular application. For example, they can be less than 1 wt. % in special bleach products, but greater than 99 wt. % in soaps or washing powder.

Particular combinations and concentrations are preferred in surfactant formulations according to the invention for particular fields of application. Thus, preferred mixtures according to the invention (detergent formulations) are those in which the proportion of linear alkylbenzenesulfonates is in the range from 7 to 10 wt. % and/or the proportion of fatty alcohol ethoxylates having 12-18 C atoms is in the range from 3 to 6 wt. %, based in each case on the total weight of the mixture. Other preferred mixtures according to the invention (formulations for light-duty detergents, shampoos and shower gels) are those in which the proportion of sodium laurylethersulfate is in the range from 7 to 13 wt. % and/or the proportion of betaine (especially betaine of the acid amide type having the structure shown above) is in the range from 1 to 3 wt. %, based in each case on the total weight of the mixture.

When surfactant formulations according to the invention are applied, the substantivity of the 2-(1,1,4-trimethylpent-3-enyl)-4,7-dihydro-1,3-dioxepin on hair and textile fibres is so pronounced as to give the impression that although the surfactants present initially bring the 2-(1,1,4-trimethylpent-3-enyl)-4,7-dihydro-1,3-dioxepin into the aqueous phase, this compound, in the presence of hair or textile fibres, is forced out of the aqueous phase and onto the hair or textile fibre. However, there is currently no scientific explanation for this observation.

The mixtures containing 2-(1,1,4-trimethylpent-3-enyl)-4,7-dihydro-1,3-dioxepin exhibit a surprisingly high substantivity or retention towards or on hair, wool, cotton and other textile fibres.

The Examples which follow serve to illustrate the invention:

### EXAMPLES

### Preliminary Remark

In the Examples below, the properties of “substantivity” and “blooming” were evaluated by a panel of experts (8-12 persons).

For the determination of substantivity, unperfumed fabric softener, shampoo or washing powder was perfumed with the conventional concentration of 2-(1,1,4-trimethylpent-3-enyl)-4,7-dihydro-1,3-dioxepin or the substances used for comparison. The olfactory assessment was made using a 7-point scale with values from 0 (no odour) to 6 (very strong odour).

For the assessment of blooming, an aqueous solution containing a low concentration of surfactant was treated with 0.1% of 2-(1,1,4-trimethylpent-3-enyl)-4,7-dihydro-1,3-dioxepin or the substances used as comparison. The evaluation was made on a 9-point scale with 1 denoting weak and 9 very strong.

### Determination of the Substantivity (Examples 1-3)

### Example 1

### Shampoo

The fragrance to be evaluated is incorporated as a 50 wt. % solution in diethyl phthalate, in a dose of 0.6 wt. %, into an unperfumed shampoo base of the following composition:

| | |
|---|---|
| Sodium laurylethersulfate | 12% |
| (e.g. Texapon NSO from Cognis Deutschland GmbH) | |
| Cocamidopropylbetaine | 2% |
| (e.g. Dehyton K from Cognis Deutschland GmbH) | |
| Sodium chloride | 1.4% |
| Citric acid | 1.3% |
| Phenoxyethanol, methyl-, ethyl-, butyl- and | 0.5% |
| propyl-paraben | |
| Water | 82.8% |

This is used to prepare 100 ml of a 20 wt. % aqueous shampoo solution (as an Example of a solution according to the invention). 2 swatches of hair are washed together for 2 minutes in this shampoo solution and then rinsed for 20 seconds under lukewarm running water. One swatch is packed wet in aluminium foil and the second swatch is dried with a hair dryer. Both swatches are assessed by a panel for their olfactory properties.

| | Evaluation of substantivity | |
|---|---|---|
| Fragrance | wet | dry |
| 2-(1,1,4-Trimethylpent-3-enyl)-4,7- | 2.8 | 1.2 |
| dihydro-1,3-dioxepin (according to the | | |
| invention) | | |
| 2-(1,1,4-Trimethylpentyl)-4,7-dihydro- | 2.2 | 0.8 |
| 1,3-dioxepin | | |
| (Example 3 of DE 195 32 318) | | |
| 2-(1,1-Dimethylpent-3-enyl)-4,7-dihydro- | 1.8 | 0.3 |
| 1,3-dioxepin | | |
| (Example 4 of DE 195 32 318) | | |
| 2-(1,1-Dimethylbut-3-enyl)-4,7-dihydro- | 2.0 | 0.5 |
| 1,3-dioxepin | | |
| (Example 5 of DE 195 32 318) | | |
| 2-(3-Methylbutyl)-2-methyl-4,7-dihydro- | 2.0 | 0.6 |
| 1,3-dioxepin | | |
| (amber sage) | | |
| 2-(1,1,4-Trimethyl-3-pentenyl)-1,3- | 1.5 | 0.1 |
| dioxane | | |

### Example 2

### Fabric Softener

The fragrance to be evaluated is incorporated as a 50 wt. % solution in diethyl phthalate, in a dose of 0.5 wt. %, into an unperfumed fabric softener base of the following composition:

| | |
|---|---|
| Quaternary ammonium methosulfate | 5.5% |
| (Esterquat), approx. 90% | |
| (e.g. Rewoquat WE 18 from Witco Surfactants GmbH) | |
| Alkyldimethylbenzylammonium chloride, | 0.2% |
| approx. 50% | |
| (e.g. Preventol R50 from Bayer AG) | |
| Dye solution, approx. 1% | 0.3% |
| Water | 94.0% |

Two cloths are rinsed for 30 minutes at 20° C. with 370 g of a 1% aqueous fabric softening liquor (as an Example of a solution according to the invention) in a Linetest machine running the fabric softener programme. The cloths are wrung out and then spun for 20 seconds. One cloth is sealed up wet and one is hung up to dry. Both cloths are then assessed by a panel for their olfactory properties.

| | Evaluation of substantivity | |
|---|---|---|
| Fragrance | wet | dry |
| 2-(1,1,4-Trimethylpent-3-enyl)-4,7- | 3.2 | 0.6 |
| dihydro-1,3-dioxepin (according to the | | |
| invention) | | |
| 2-(1,1,4-Trimethylpentyl)-4,7-dihydro- | 2.4 | 0.3 |
| 1,3-dioxepin | | |
| (Example 3 of DE 195 32 318) | | |
| 2-(1,1-Dimethylpent-3-enyl)-4,7-dihydro- | 2.4 | 0.3 |
| 1,3-dioxepin | | |
| (Example 4 of DE 195 32 318) | | |
| 2-(1,1-Dimethylbut-3-enyl)-4,7-dihydro- | 2.0 | 0.1 |
| 1,3-dioxepin | | |
| (Example 5 of DE 195 32 318) | | |
| 2-(3-Methylbutyl)-2-methyl-4,7-dihydro- | 2.1 | 0.2 |
| 1,3-dioxepin | | |
| (amber sage) | | |
| 2-(1,1,4-Trimethyl-3-pentenyl)-1,3- | 1.6 | 0.1 |
| dioxane | | |

### Example 3

### Washing Powder

The fragrance to be evaluated is incorporated as a 50 wt. % solution in diethyl phthalate, in a dose of 0.4 wt. %, into an unperfumed washing powder base of the following formulation:

| | |
|---|---|
| Linear Na alkylbenzenesulfonate | 8.8% |
| Ethoxylated C12-18 fatty alcohol (7 EO) | 4.7% |
| Na soap | 3.2% |
| Antifoam | 3.9% |
| (Dow Corning ® 2-4248S Powdered Antifoam, | |
| silicone oil on zeolite as carrier) | |
| Zeolite 4A | 28.3% |
| Na carbonate | 11.6% |
| Na salt of an acrylic acid/maleic acid | |
| copolymer | 2.4% |
| (Sokalan CP5) | |
| Na silicate | 3.0% |
| Carboxymethyl cellulose | 1.2% |
| Dequest 2066 | 2.8% |
| ([[(phosphonomethyl)imino]bis[(ethylene- | |
| nitrilo)bis(methylene)]]tetrakisphosphonic | |
| acid, sodium salt) | |
| Optical brightener | 0.2% |
| Na sulfate | 6.5% |
| Protease | 0.4% |
| Sodium perborate tetrahydrate | 22.0% |
| TAED | 1.0% |

Two cloths are washed for 45 minutes at 60° C. with 370 g of a 1% aqueous washing powder liquor (as an Example of a solution according to the invention) in a Linetest machine running the main washing cycle. The cloths are first rinsed for 5 minutes with cold water, wrung out and then spun for 20 seconds. One cloth is sealed up wet and one is hung up to dry. Both cloths are then assessed by a panel for their olfactory properties.

| | Evaluation of substantivity | |
|---|---|---|
| Fragrance | wet | dry |
| 2-(1,1,4-Trimethylpent-3-enyl)-4,7- | 2.8 | 1.3 |
| dihydro-1,3-dioxepin (according to the | | |
| invention) | | |
| 2-(1,1,4-Trimethylpentyl)-4,7-dihydro- | 2.2 | 1.0 |
| 1,3-dioxepin | | |
| (Example 3 of DE 195 32 318) | | |
| 2-(1,1-Dimethylpent-3-enyl)-4,7-dihydro- | 1.9 | 0.6 |
| 1,3-dioxepin | | |
| (Example 4 of DE 195 32 318) | | |
| 2-(1,1-Dimethylbut-3-enyl)-4,7-dihydro- | 2.0 | 0.7 |
| 1,3-dioxepin | | |
| (Example 5 of DE 195 32 318) | | |
| 2-(3-Methylbutyl)-2-methyl-4,7-dihydro- | 2.0 | 0.6 |
| 1,3-dioxepin | | |
| (amber sage) | | |
| 2-(1,1,4-Trimethyl-3-pentenyl)-1,3- | 1.5 | 0.2 |
| dioxane | | |

### Determination of the Blooming (Example 4)

### Example 4

0.1 g of fragrance is mixed with 2 g of a neutral surfactant (Cremofor C0445). This mixture is diluted with 98 g of water. The odour of this solution is assessed by a panel on a scale of 1-9 from an open 250 ml glass beaker using decanol=4 and hyacinthene body=7 as reference materials.

| Fragrance | Evaluation of blooming |
|---|---|
| 2-(1,1,4-Trimethylpent-3-enyl)-4,7- | 7 |
| dihydro-1,3-dioxepin (according to the | |
| invention) | |
| 2-(1,1,4-Trimethylpentyl)-4,7-dihydro- | 4 |
| 1,3-dioxepin | |
| (Example 3 of DE 195 32 318) | |
| 2-(1,1-Dimethylpent-3-enyl)-4,7-dihydro- | 3 |
| 1,3-dioxepin | |
| (Example 4 of DE 195 32 318) | |
| 2-(1,1-Dimethylbut-3-enyl)-4,7-dihydro- | 3 |
| 1,3-dioxepin | |
| (Example 5 of DE 195 32 318) | |
| 2-(3-Methylbutyl)-2-methyl-4,7-dihydro- | 4 |
| 1,3-dioxepin | |
| (amber sage) | |
| 2-(1,1,4-Trimethyl-3-pentenyl)-1,3- | 2 |
| dioxane | |

### Fragrance Compositions

### Example 5

Fragrance composition, particularly suitable for use in shampoos, consisting of:

| | Fragrance mixture I | Fragrance mixture II |
|---|---|---|
| | parts by weight | parts by weight |
| Aldehyde C8 10% DPG | 2 | 2 |
| Aldehyde C12 lauric | 3.5 | 3.5 |
| Hexenal trans-2 | 9 | 9 |
| 1% DPG | | |
| Hexenol cis-3 | 2 | 2 |
| Hexenyl acetate cis-3 | 4 | 4 |
| Vertocitral | 7.4 | 7.4 |
| Allylamyl glycolate | 0.5 | 0.5 |
| Dihydromyrcenol | 46 | 46 |
| Lemongrass oil rect. | 1 | 1 |
| Geranonitrile 10% DPG | 2.8 | 2.8 |
| Citrylal | 0.5 | 0.5 |
| Orange oil 5x | 25 | 25 |
| Claritone ® | 5 | 5 |
| Methyl anthranilate | 1 | 1 |
| 10% DPG | | |
| Hexyl acetate | 5 | 5 |
| Isoamyl acetate 10% DPG | 2 | 2 |
| Jasmaprunate | 4 | 4 |
| Prenyl acetate | 4 | 4 |
| Ethyl heptylate | 9 | 9 |
| Aldehyde C14 known as | 15 | 15 |
| Extra | | |
| Decalactone gamma | 4 | 4 |
| Ethyl 2-methylbutyrate | 5 | 5 |
| Manzanate 10% DPG | 4 | 4 |
| Allylcyclohexyl propionate | 3 | 3 |
| Allyl heptylate | 20 | 20 |
| Wine yeast oil green 10% | 3 | 3 |
| DPG | | |
| Peach #D40110PM | 6 | 6 |
| Davana oil for perfume | 0.5 | 0.5 |
| Blackcurrant #DB10002 | 6 | 6 |
| Aldehyde C16 known as P | 0.5 | 0.5 |
| Lilial ® | 65 | 65 |
| Helional ® | 4 | 4 |
| Lyral ® | 6 | 6 |
| Linalool | 40 | 40 |
| Dimethylbenzylcarbinyl | 6 | 6 |
| butyrate | | |
| Tagetes oil BM | 0.5 | 0.5 |
| Phenirat ® | 160 | 160 |
| Citronellol 950 | 18 | 18 |
| Geranyl acetate pure | 3 | 3 |
| Damascone delta | 1.2 | 1.2 |
| Rose booster #D50221A | 5 | 5 |
| 10% DPG | | |
| Benzyl acetate | 25 | 25 |
| Hexylcinnamaldehyde alpha | 90 | 90 |
| Jasmin cis | 3 | 3 |
| Jasmin # 151 | 4 | 4 |
| Benzyl salicylate | 85 | 85 |
| Isoamyl salicylate | 30 | 30 |
| Ionone beta | 55 | 55 |
| Irolene 1% DPG | 1 | 1 |
| Isoeugenol | 0.8 | 0.8 |
| Anisaldehyde pure | 2.5 | 2.5 |
| Vanillin | 3 | 3 |
| Cinnamon oil 10% DPG | 3 | 3 |
| Agrumex HC | 68 | 68 |
| Herbyl propionate | 24 | 24 |
| Vertofix BM | 80 | 80 |
| Sandolen H&R ® | 10.5 | 10.5 |
| Evernyl | 1 | 1 |
| Ambrinol S 10% DPG | 1.5 | 1.5 |
| Globalide ® 100% | 90 | 90 |
| Macrolide Supra 50% in TEC | 33.3 | 33.3 |
| 2-(1,1,4-Trimethylpentyl)- | 80.0 | — |
| 4,7-dihydro-1,3-dioxepin | | |
| (comparative substance) | | |
| 2-(1,1,4-Trimethylpent-3- | — | 80.0 |
| enyl)-4,7-dihydro-1,3- | | |
| dioxepin (according to the | | |
| invention) | | |

A hair washing test was carried out as indicated in Example 1 using fragrance mixture I or fragrance mixture II to perfume the shampoo.

Evaluation of blooming

| Fragrance | wet | dry |
|---|---|---|
| Fragrance composition I | 2.3 | 1.2 |
| Fragrance composition II | 3.0 | 1.6 |
| (according to the invention) | | |

Apart from improved substantivity, the swatches washed with fragrance mixture II (according to the invention, i.e. with 2-(1,1,4-trimethylpent-3-enyl)-4,7-dihydro-1,3-dioxepin) exhibited a markedly fresher green-flowery top note, which was also characterized by chrysanthemum and grapefruit aspects.

1. A process for providing (a) hair or (b) textile fibres with a fresh smell with steps that include contacting said hair or fibres with 2-(1,1,4-trimethylpent-3-enyl)-4,7-dihydro-1,3-dioxepin.

2. A process according to claim 1 wherein said 2-(1,1,4-trimethylpent-3-enyl)-4,7-dihydro-1,3-dioxepin is present in an amount sufficient for efficacy as an agent for increasing the substantivity and/or retention of a fragrance mixture.

3. A process according to claim 1 for providing (a) hair or (b) textile fibres with a fresh smell, comprising the following steps:
preparation of a mixture containing 2-(1,1,4-trimethylpent-3-enyl)-4,7-dihydro-1,3-dioxepin, and
application of the mixture to the hair or textile fibres.

4. A composition useful for enhancing the substantivity or retention of a fragrance said composition comprising
water
2-(1,1,4-trimethylpent-3-enyl)-4,7-dihydro-1,3-dioxepin and
one or more surfactants,
the concentration of 2-(1,1,4-trimethylpent-3-enyl)-4,7-dihydro-1,3-dioxepin in the solution being in the range from 10⁻⁷ to 10⁻¹ wt. %.

5. A composition according to claim 4 wherein said 2-(1,1,4-trimethylpent-3-enyl)-4,7-dihydro-1,3-dioxepin is present in an amount effective for increasing the odour perceived above an aqueous solution containing surfactants.

6. Mixture comprising 2-(1,1,4-trimethylpent-3-enyl)-4,7-dihydro-1,3-dioxepin and one or more surfactants selected from the group comprising linear alkyl-benzenesulfonates, fatty alcohol ethoxylates having 12-18 C atoms, laurylethersulfates and betaines.

7. Mixture according to claim 6 wherein the proportion of linear alkylbenzenesulfonates is in the range from 7 to 10 wt. % and/or the proportion of fatty alcohol ethoxylates having 12-18 C atoms is in the range from 3 to 6 wt. %, based in each case on the total weight of the mixture.

8. Mixture according to claim 6 wherein the proportion of sodium laurylethersulfate is in the range from 7 to 13 wt. % and/or the proportion of betaine is in the range from 1 to 3 wt. %, based in each case on the total weight of the mixture.

9. A process for increasing substantivity and/or retention of a fragrance mixture associated with hair or textile fibres that comprises:
adding 2-(1,1,4-trimethylpent-3-enyl)-4,7-dihydro-1,3-dioxepin to a fragrance mixture to form an enhanced mixture; and
contacting said enhanced mixture with hair or textile fibres.

10. A process according to claim 9 wherein said fragrance mixture comprises a shampoo for hair.

11. A process according to claim 9 wherein said fragrance mixture comprises a detergent for textile fibres.

12. A process according to claim 9 wherein said 2-(1,1,4-trimethylpent-3-enyl)-4,7-dihydro-1,3-dioxepin is present in said enhanced mixture in an amount within the range from 10⁻⁷ to 10⁻¹ wt. %.

13. A process according to claim 9 wherein said enhanced mixture comprises a surfactant selected from the group comprising linear alkylbenzenesulfonates, fatty alcohol ethoxylates having 12-18 C atoms, laurylethersulfates and betaines.

14. A process according to claim 9 wherein said 2-(1,1,4-trimethylpent-3-enyl)-4,7-dihydro-1,3-dioxepin is present in said enhanced mixture in an amount sufficient to impart a pronounced fresh-green top note.

15. A process according to claim 14 wherein said 2-(1,1,4-trimethylpent-3-enyl)-4,7-dihydro-1,3-dioxepin is present in said enhanced mixture in an amount sufficient to impart a blooming odor perceived above an aqueous solution containing surfactants.
